(19) United States
(12) Patent Application Publication
(10) Pub. No.: US 2024/0299422 A1
(43) Pub. Date: Sep. 12, 2024

(54) Topical Cannabinoid Compositions and Methods for Treating Skin Diseases

(71) Applicant: Avicanna Inc., Toronto (CA)

(72) Inventors: Aras Azadian, Toronto (CA); Satish Asotra, Brampton (CA); AKM Abdul Hai, Etobicoke (CA)

(73) Assignee: Avicanna Inc., Toronto, ON (CA)

(21) Appl. No.: 18/549,878

(22) PCT Filed: Mar. 7, 2022
(86) PCT No.: PCT/IB2022/052014

§ 371 Date: Sep. 8, 2023

Related U.S. Application Data
(60) Provisional application No. 63/157,948, filed on Mar. 8, 2021.

(51) Int. Cl. A61K 31/00; A61K 9/00; A61K 9/06; A61K 47/10

(52) U.S. Cl. CPC A61K 31/658; A61K 9/0014; A61K 9/06; A61K 47/10

(57) ABSTRACT

Topical compositions comprising cannabinoids in combination with one or more humectants and one or more penetration enhancers are provided. Also provided are methods comprising applying a topical cannabinoid composition provided herein to the skin of a subject and uses of a topical cannabinoid formulation provided herein for the treatment of a skin disease or condition in a subject.

### FIELD

The present disclosure relates to compositions comprising one or more cannabinoids for topical application.

### BACKGROUND

There is a growing number of patients suffering from skin diseases, that are seeking natural remedies as alternative or complementary therapy. Such skin diseases range from simple rashes that occur in combination with itching and redness, to chronic conditions such as eczema, rosacea, seborrheic dermatitis, psoriasis and epidermolysis bullosa.

Currently the most effective and commonly used prescription drugs for treating skin diseases are oral and topical corticosteroids, particularly glucocorticoid related steroids. They are very effective for many forms of eczema, including atopic dermatitis, and are fairly effective in ameliorating the symptoms of psoriasis. But these corticosteroids are safe for only short periods of time because they exert some negative side effects on skin, including suppression of the skin's immunity; hyperglycemia; anti-proliferative/thinning effect on the skin; and adrenal gland impairment. Also, these corticosteroids are not particularly effective in treating acute diseases, like UV-induced sunburn.

Systemic medications are usually used for patients with moderate to severe skin diseases. Several agents, methotrexate and 6-thioguanine, are actually chemotherapeutics used for cancer indications. These drugs are both hepatotoxic and teratogenic. Bone marrow suppression and anemia are the major complications associated with these agents that can occur at any time during treatment.

Other systemic agents include retinoids which can cause severe, life-threatening birth defects, psychiatric symptoms and hepatotoxicity. Examples of retinoids include isotretinoin and acitretin. Additional systemics include cyclosporin, sulfasalazine and mycophenolate mofetil (MMF). All these drugs have numerous drug interaction issues.

Recently, biologics such as infliximab, efalizumab, adalimumab, etanercept and alefacept have been used. Each of these agents requires painful injections or infusions which must be either given in a physician's office or self-administered by patients themselves.

Immunosuppressant agents are available, but their long-term effects are not known. Serious infections, cardiovascular events and increased risk of malignancies are the greatest health concerns with immunosuppressant agents.

Apart from the above drawbacks, none of the above therapies are specifically targeted to treat itching associated with the skin diseases. Furthermore, no cure exists for diseases like epidermolysis bullosa, a rare genetic disorder in which disease progresses from nuisance to disability. Management of epidermolysis bullosa includes supportive care to protect the skin from blistering, and prevention and treatment of secondary infections. Typically, such treatment is similar to that given to burn victims, and may include application of non-adherent bandages, dressings, and antiseptic washes for the blisters, various medications for pain, itching, and inflammation, and oral antibiotics for infections.

With the discovery of a class of cannabinoid receptors and their involvement in diseases, attempts have been made at orally administering compounds which act on these cannabinoid receptors in the form of a capsule. Such compounds include phytocannabinoids and synthetic cannabinoids. However, patients with severe nausea are often not able to retain the capsule in their stomachs long enough for the cannabinoids to take effect.

Another issue with capsules, as well as smoked cannabis, is that patients absorb cannabinoids relatively rapidly (as compared to controlled drug delivery rates) and receive high cannabinoid concentrations in their body. These high concentrations, or peak levels, are often associated with serious psychoactive and other central nervous system side effects.

Due to the side effects associated with oral and systemic administration of active agents, there is a continuing need to develop and provide safe treatments for dermatological indications, which are derived, at least in part, from natural sources. Additionally, there is a particular need to treat the symptoms of skin diseases, with a specific focus on healing blisters, alleviating itching and reducing inflammation in the course of the skin diseases through topical application of active agents. In particular such active agents applied topically should exert less negative side effects than oral and systemic applications.

With relaxation of laws regulating cannabis use, there now exists the opportunity to explore the potential of cannabinoids for treating skin diseases. Few studies reported therapeutic potential of the two major cannabinoids present in cannabis, namely the psychoactive compound tetrahydrocannabinol (THC) and the non-psychoactive compound cannabidiol (CBD), for the treatment of skin diseases.

### SUMMARY

In one aspect, there is provided a topical cannabinoid composition comprising:
a. a cannabinoid at 0.01-10% (w/w),
b. a humectant at 0.01-10% (w/w),
c. a penetration enhancer at 1-5% (w/w), and
d. water to make up 100% by weight,
   wherein the topical cannabinoid composition comprises less than 40% (w/w) of ethanol.

In an embodiment of the topical cannabinoid composition as described herein, the topical cannabinoid composition comprises no more than 10% (w/w) of ethanol.

In an embodiment of the topical cannabinoid composition as described herein, the topical cannabinoid composition comprises no more than 1% (w/w) of ethanol.

In another aspect, there is provided a topical cannabinoid composition comprising:
a. a cannabinoid at 0.01-10% (w/w),
b. a humectant at 0.01-10% (w/w),
c. a penetration enhancer at 1-5% (w/w), and
d. water at no less than 31% (w/w).

In an embodiment of the topical cannabinoid composition as described herein, at least one of the humectant and the penetration enhancer has a molecular weight lower than the molecular weight of the cannabinoid.

In an embodiment of the topical cannabinoid composition as described herein, the humectant is propylene glycol, and the penetration enhancer is diethylene glycol monoethyl ether.

In an embodiment of the topical cannabinoid composition as described herein, the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), or any combination thereof.

In an embodiment of the topical cannabinoid composition as described herein, the cannabinoid is cannabidiol (CBD).

In an embodiment of the topical cannabinoid composition as described herein, the composition further comprises a thickening agent at 1-10% (w/w).

In an embodiment of the topical cannabinoid composition as described herein, the thickening agent is cetostearyl alcohol.

In an embodiment of the topical cannabinoid composition as described herein, the composition has a pH of 5-7.

In an embodiment of the topical cannabinoid composition as described herein, the composition further comprises one or more of:
a. a first emulsifier at 0.5-5% (w/w),
b. a skin protectant at 1-15% (w/w),
c. an emollient at 1-5% (w/w),
d. a preservative at 0.1-3% (w/w),
e. a sunscreen agent at 0.1-0.5% (w/w),
f. a second emulsifier at 1-5% (w/w),
g. a pH adjusting agent in a quantity sufficient for the composition to maintain a pH of 5-7, and
h. water to make up 100% by weight.

In an embodiment of the topical cannabinoid composition as described herein, the thickening agent is cetostearyl alcohol, the first emulsifier is glycerol monostearate, the skin protectant is white petrolatum, the emollient is caprylic/capric triglyceride, the preservative is phenoxyethanol, the sunscreen agent is titanium dioxide, the second emulsifier is Polysorbate 60, and the pH adjusting agent is citrate/citric acid.

In an embodiment of the topical cannabinoid composition as described herein, the composition is a cream, ointment, gel, lotion, liquid, solution, spray, aerosol, any other dosage form suitable for topical application, or any combination thereof.

In an embodiment of the topical cannabinoid composition as described herein, the composition is a cream.

In another aspect, there is provided a cream comprising:
a. a cannabinoid at 0.01-10% (w/w),
b. a cannabinoid localizing agent at 1-15% (w/w), and
c. water to make up 100% by weight,
In an embodiment of the cream as described herein, the composition has a viscosity in a range of about 20,000 to about 100,000 cps.

In another aspect, there is provided a method comprising applying the topical cannabinoid composition as described herein to the skin of a subject.

In an embodiment of the method as described herein, the skin of the subject is affected by a skin disease or condition.

In an embodiment of the method as described herein, the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; skin cancer; squamous cell carcinoma; Sweet's syndrome; vitiligo; or, any symptom or side effect associated with any of said skin diseases or conditions. In another aspect, there is provided use of the topical cannabinoid formulation described herein for the treatment of a skin disease or condition in a subject.

In an embodiment of the use as described herein, the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoictic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacca; scabies; shingles; skin cancer; squamous cell carcinoma; Sweet's syndrome; vitiligo; or, any symptom or side effect associated with any of said skin diseases or conditions.

Other aspects, features, and embodiments of the present disclosure will become apparent to those of ordinary skill in the art upon review of the following description of specific embodiments in conjunction with the accompanying figures.

### BRIEF DESCRIPTION OF THE FIGURES

Compositions provided herein, in accordance with one or more various embodiments, are described in detail with reference to the following figures. The drawings are provided for the purposes of illustration only and merely depict typical or example embodiments of the disclosed compositions.

FIG. 1 illustrates diffusion rates of different CBD cream lots through 0.45 μm Whatman™ cellulose acetate synthetic membrane.

FIG. 2 illustrates CBD diffusion rates through EMD Millipore Strat-M™ membrane in sustained release manner.

FIG. 3 illustrates a typical chromatogram of the analysis of CBD (50 μg/mL) using isocratic method 20:80 (water:acetonitrile) with 0.1% TFA—1 mL/min.

FIG. 4 illustrates CBD quantification validation through calibration curves.

FIG. 5 illustrates plasma profile of CBD following twice daily application of the topical CBD composition of Example 1 (n=12) in rats. Red line denotes the limit of quantitation (LOQ) of the LC-MS method (i.e., 5 ng/ml).

FIG. 6 illustrates percentage weight changes of the test animals over a 7-day period following twice-daily application of the topical CBD composition of Example 1 (n=12).

FIG. 7 illustrates representative images of the application site for the topical CBD composition of Example 1 at T=168 hours. The area of rat skin enclosed in the red dashed circle denotes mild skin irritation/pinching as a result of the use of dermal application jackets.

### DETAILED DESCRIPTION

It has been surprisingly discovered by the present inventors that the cannabinoid or combination of cannabinoids comprised in compositions provided herein, upon topical application, is available at the site of administration in a mammal in a therapeutically effective amount but is not absorbed systemically in a therapeutically effective concentration.

Compositions provided herein comprise the combination of humectants and penetration enhancers, which are selected such that these ingredients not only improve penetration of cannabinoids into the epidermis, but they also enter the sub-epidermal layers (such as dermis and hypodermis layers) quickly with the resulting effects that transportation/diffusion of cannabinoids through the sub-epidermal layers of the skin is reduced.

It is expected that compositions provided herein would have less frequency and severity of side-effects associated with oral and systemic cannabinoid administration because the amount of cannabinoids circulating in a subject should be reduced due to first bypass of portal circulation.

Provided herein are topical compositions comprising cannabinoids which penetrate into the epidermis of the skin and remain there following application to the skin for a sufficient time to be absorbed and have a therapeutic effect locally, e.g., through skin.

Compositions provided herein may be useful for treating skin diseases and conditions including, but not limited to, eczema, psoriasis, epidermolysis bullosa and skin cancer. Compositions provided herein may also be useful for protecting skin or enhancing the appearance of skin.

Compositions provided herein can also be used in conjunction with available treatments of skin diseases.

Topical cannabinoid compositions provided herein exhibit excellent overall stability and viscosity.

“Cannabinoid,” as used herein, is meant to include compounds which interact with the cannabinoid receptor and various cannabinoid mimetics, such as cannabidiol (CBD), cannabidiolic acid (CBDA), cannabidivarin (CBDV), cannabigerol (CBG), cannabigervarin (CBGV), tetrahydrocannabivarin (THCV) and tetrahydrocannabinol (THC).

“Phytocannabinoids” as used herein means cannabinoids extracted from Cannabis plant species including by the way of non-limiting example Cannabis sativa, Cannabis indica and Cannabis ruderalis and all resins, stalks, flowers, seeds and oils related thereto.

The term “active agent” is generally understood to mean an active pharmaceutical ingredient.

The terms “topical composition” or “topical formulation” mean a composition in which an active agent may be placed for direct application to a skin surface and from which a therapeutically effective amount of the active agent may be released. Such formulations may include creams, ointments, gels, lotions, or any other dosage form suitable for topical application.

The terms “skin” or “skin surface” is meant to include the outer skin of a subject comprising one or more epidermal layers.

“Cream” means a liquid or semi-liquid colloid at ambient temperature wherein the dispersed phase is dispersed in a liquid/semi-liquid continuous medium. A cream is more viscous than a liquid but less viscous than a gel.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require. It will be understood that a therapeutically and/or prophylactically effective amount of an active agent for a subject is dependent inter alia on the body weight of the subject as well as other factors known to a person of ordinary skill in the art.

The terms “treat,” “treating,” or “treatment of” are used herein in their broad senses unless otherwise specifically indicated in the particular context, and results of a treatment may generally include reversing, alleviating, or inhibiting the progress of an indicated disease or condition, or one or more symptoms of the disease or condition.

“wt %” or “w/w %” when referring to the percentage of a component in a composition is percentage of the weight of the component in the composition relative to the total weight of the composition.

The term “excipient” herein means any substance, not itself an active agent, which may be used as a carrier or vehicle for delivery of an active agent to a subject or combined with an active agent to improve its handling or storage properties or to permit or facilitate formation of a dose unit of the composition. Examples of excipients include, but are not limited to, a “solubility agent” or “solubilizing agent”, which is added to a composition to increase the solubility of a solute; an “emulsifier” or “emulsifying agent”, which is capable of lowering surface tension between a non-polar and polar phase; a “humectant”, which is capable of attracting or retaining moisture; a “penetration enhancer”, which is capable of improving penetration of an active agent into the epidermis; a “thickening agent”, which is capable of increasing the viscosity of a composition or formulation; a “skin protectant” or “skin protecting agent”, which is capable of temporarily protecting injured or exposed skin from harmful or irritating stimuli, thereby reducing discomfort associated with such skin; and a “stability agent”, which is added to a composition to improve its stability under conditions such as weekly cycling between freezer and ambient room temperature.

As used herein, the term “stable,” when referring to a composition, means that the composition can be cycled weekly between freezer and ambient room temperature conditions for a minimum of 1 month while retaining its pH and viscosity within defined ranges.

“Alleviate” as used herein, is meant to include complete elimination as well as any clinically or quantitatively measurable reduction in the subject's symptoms and/or discomfort.

A “subject” herein to which a therapeutic agent or composition thereof can be administered includes mammals such as a human subject of either sex and of any age, and also includes any nonhuman animal, particularly a domestic or companion animal such as a cat, dog or horse, as well as laboratory animals such as guinea pigs.

As discussed in greater detail in the illustrative and non-limiting examples provided herein, the present disclosure is directed to topical formulations/compositions that incorporate at least one cannabinoid.

In one aspect, there is provided a topical cannabinoid composition comprising:
a. a cannabinoid at 0.01-10% (w/w),
b. a humectant at 0.01-10% (w/w),
c. a penetration enhancer at 1-5% (w/w), and
d. water to make up 100% by weight,
   wherein the topical cannabinoid composition comprises less than 40% (w/w) of a lower alcohol.

As used herein, the term “lower alcohol” means any alcohol containing fewer than five carbons, such as ethanol, isopropanol, ethylene glycol, or propylene glycol. As used herein, a concentration of a component referred herein is a total concentration when a topical cannabinoid composition provided herein comprises two or more compounds that are considered to belong to the same component (e.g., for a topical cannabinoid composition comprising a mixture of tetrahydrocannabinol and cannabidiol, both cannabinoids, 5% (w/w) of a cannabinoid is the total concentration of the mixture of tetrahydrocannabinol and cannabidiol).

In certain exemplary, non-limiting embodiments, a topical cannabinoid composition provided herein comprises less than 35%, less than 30%, less than 25%, less than 20%, less than 15%, less than 10%, less than 5%, less than 1%, less than 0.5%, or less than 0.1% of the lower alcohol.

In another aspect, there is provided a topical cannabinoid composition comprising:
a. a cannabinoid at 0.01-10% (w/w),
b. a humectant at 0.01-10% (w/w),
c. a penetration enhancer at 1-5% (w/w), and
d. water at no less than 31% (w/w).

In certain exemplary, non-limiting embodiments, a topical cannabinoid composition provided herein comprises water at no less than 35% (w/w), 40% (w/w), 45% (w/w), 50% (w/w), 55% (w/w), 60% (w/w), 65% (w/w), 70% (w/w), 75% (w/w), 80% (w/w), 85% (w/w), 90% (w/w), 91% (w/w), 92% (w/w), 93% (w/w), 94% (w/w), 95% (w/w), 96% (w/w), 97% (w/w), or 98% (w/w).

In another aspect, there is provided a cream comprising:
a. a cannabinoid at 0.01-10% (w/w),
b. a cannabinoid localizing agent at 1-15% (w/w), and
c. water to make up 100% by weight,
As used herein, the term “cannabinoid localizing agent” includes one or more compounds or materials that can move through the epidermis of skin to reach the sub-epidermal layers more quickly than a cannabinoid in the cream when the cream is applied to the skin, and can obstruct, reduce or slow down movement of the cannabinoid into and through the sub-epidermal layers, thereby reducing transportation/diffusion of the cannabinoid through the sub-epidermal layers. In an embodiment, a combination of a humectant and a penetration enhancer can serve as a cannabinoid localizing agent when the humectant and penetration enhancer are selected such that the combination not only improves penetration of cannabinoids into the epidermis, but also enters the sub-epidermal layers (such as dermis and hypodermis layers) quickly with the resulting effects that transportation/diffusion of cannabinoids through the sub-epidermal layers of the skin is reduced. In another embodiment, cannabinoid localizing agents include a combination of a humectant and a penetration enhancer selected as described above, further in combination with a thickening agent selected for localizing the cannabinoid in or on the epidermis. In a further embodiment, a cannabinoid localizing agent includes a plugging agent that can plug pores in the sub-epidermal layers to obstruct passage of cannabinoid molecules through these layers.

Exemplary cannabinoids include cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), combinations, and mixtures thereof extracted from Cannabis plant species including Cannabis sativa, Cannabis indica and Cannabis ruderalis and all resins, stalks, flowers, seeds and oils related thereto.

In certain exemplary, non-limiting embodiments, a topical cannabinoid composition provided herein may include about 0.01% to about 10% (w/w) of cannabinoid(s). For example, a topical cannabinoid composition provided herein may comprise about 0.1% to about 10%, about 0.5% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.01% to about 9%, about 0.1% to about 9%, about 0.5% to about 9%, about 1% to about 9%, about 2% to about 9%, about 3% to about 9%, about 4% to about 9%, about 5% to about 9%, about 6% to about 9%, about 7% to about 9%, about 8% to about 9%, about 0.01% to about 8%, about 0.1% to about 8%, about 0.5% to about 8%, about 1% to about 8%, about 2% to about 8%, about 3% to about 8%, about 4% to about 8%, about 5% to about 8%, about 6% to about 8%, about 7% to about 8%, about 0.01% to about 7%, about 0.1% to about 7%, about 0.5% to about 7%, about 1% to about 7%, about 2% to about 7%, about 3% to about 7%, about 4% to about 7%, about 5% to about 7%, about 6% to about 7%, about 0.01% to about 6%, about 0.1% to about 6%, about 0.5% to about 6%, about 1% to about 6%, about 2% to about 6%, about 3% to about 6%, about 4% to about 6%, about 5% to about 6%, about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.1% to about 1%, or about 0.5% to about 1% (w/w) of cannabinoid(s).

In certain exemplary, non-limiting embodiments, a topical cannabinoid composition provided herein may comprise at least 0.01%, at least 0.1%, at least 0.5%, at least 1%, at least 2%, at least 3%, at least 4%, at least 5%, at least 6%, at least 7%, at least 8%, at least 9% or at least 10% (w/w) of cannabinoid(s).

In certain exemplary, non-limiting embodiments, a topical cannabinoid composition provided herein may comprise about 0.01%, about 0.1%, about 0.5%, about 1%, about 2%, about 3%, about 4%, about 5%, about 6%, about 7%, about 8%, about 9% or about 10% (w/w) of cannabinoid(s).

In certain exemplary, non-limiting embodiments, topical cannabinoid compositions provided herein may be provided as creams, ointments, gels, liquids, lotions, solutions, sprays, aerosols, or combinations thereof. In certain exemplary, non-limiting embodiments, a topical cannabinoid composition provided herein may have a viscosity in a range of about 10,000 to about 250,000 cps.

In certain exemplary, non-limiting embodiments, topical cannabinoid compositions provided herein may be provided as creams. Creams offer ease of application, least irritation to the skin and optimal viscosity (e.g., in a range of about 20,000 to about 100,000 cps, about 30,000 to about 100,000 cps, about 40,000 to about 100,000 cps, about 50,000 to about 100,000 cps, about 60,000 to about 100,000 cps, or about 70,000 to about 100,000 cps) that is needed for prolonged residence time of cannabinoids on the epidermis of the skin, thereby providing beneficial effects on the impacted tissue or the tissue of interest for amelioration of skin disease conditions.

Without being limited by any particular theory, it is expected that creams having higher viscosity would stay for a longer time on the skin (longer residence time), facilitating optimal absorption of the active agent. Further, the thickness of a cream may help maintain the integrity of the composition of the applied cream on the epidermis for extended time periods without drying.

In certain exemplary, non-limiting embodiments, a topical cannabinoid composition may include cannabinoids in a specific therapeutic amount for treating subjects suffering from a skin disease or condition such as: microbial infection-induced dermatitis; solar dermatitis; atopic dermatitis; contact dermatitis; acne; alopecia areata; basal cell carcinoma; Bowen's disease; congenital erythropoictic porphyria; Darier's disease; epidermolysis bullosa; eczema; erythropoictic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; skin cancer; squamous cell carcinoma; Sweet's syndrome; vitiligo; or, any symptom or side effect associated with any of said skin diseases or conditions.

Other ingredients may be provided in topical cannabinoid compositions provided herein, so long as they are physiologically acceptable and suitable for use in combination with cannabinoids.

In certain exemplary, non-limiting embodiments, a topical cannabinoid composition provided herein may include one or more humectants, which may be a polyol and/or have a molecular weight lower than that of the cannabinoid(s) comprised in the topical cannabinoid composition. Examples of humectants include glycerin, propylene glycol, butylene glycol, dipropylene glycol, pentylene glycol, hexylene glycol, polyethylene glycol, combinations, and mixtures thereof.

In certain exemplary, non-limiting embodiments, a topical cannabinoid composition provided herein may include about 0.01% to about 10% (w/w) of humectant(s). For example, a topical cannabinoid composition provided herein may comprise about 0.1% to about 10%, about 0.5% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.01% to about 9%, about 0.1% to about 9%, about 0.5% to about 9%, about 1% to about 9%, about 2% to about 9%, about 3% to about 9%, about 4% to about 9%, about 5% to about 9%, about 6% to about 9%, about 7% to about 9%, about 8% to about 9%, about 0.01% to about 8%, about 0.1% to about 8%, about 0.5% to about 8%, about 1% to about 8%, about 2% to about 8%, about 3% to about 8%, about 4% to about 8%, about 5% to about 8%, about 6% to about 8%, about 7% to about 8%, about 0.01% to about 7%, about 0.1% to about 7%, about 0.5% to about 7%, about 1% to about 7%, about 2% to about 7%, about 3% to about 7%, about 4% to about 7%, about 5% to about 7%, about 6% to about 7%, about 0.01% to about 6%, about 0.1% to about 6%, about 0.5% to about 6%, about 1% to about 6%, about 2% to about 6%, about 3% to about 6%, about 4% to about 6%, about 5% to about 6%, about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.1% to about 1%, or about 0.5% to about 1% (w/w) of humectant(s).

In certain exemplary, non-limiting embodiments, a topical cannabinoid composition provided herein may comprise at least 0.01%, at least 0.1%, at least 0.5%, at least 1%, at least 2%, at least 3%, at least 4%, at least 5%, at least 6%, at least 7%, at least 8%, at least 9% or at least 10% (w/w) of humectant(s).

In certain exemplary, non-limiting embodiments, a topical cannabinoid composition provided herein may comprise about 0.01%, about 0.1%, about 0.5%, about 1%, about 2%, about 3%, about 4%, about 5%, about 6%, about 7%, about 8%, about 9% or about 10% (w/w) of humectant(s).

In certain exemplary, non-limiting embodiments, a topical cannabinoid composition provided herein may include one or more penetration enhancers which may be a glycol ether, octyldecanol, or isopropyl myristate, and/or have a molecular weight lower than that of the cannabinoid(s) comprised in the topical cannabinoid composition. Examples of penetration enhancers include diethylene glycol monoethyl ether, steareth-20, steareth-2, octyldecanol, isopropyl myristate, combinations, and mixtures thereof.

In certain exemplary, non-limiting embodiments, a topical cannabinoid composition provided herein may include about 1% to about 5% (w/w) of penetration enhancer(s). For example, a topical cannabinoid composition provided herein may comprise about 2% to about 5%, about 3% to about 5%, about 4% to about 5%, about 1% to about 4%, about 2% to about 4%, about 3% to about 4%, about 1% to about 3%, about 2% to about 3%, or about 1% to about 2% (w/w) of penetration enhancer(s).

In certain exemplary, non-limiting embodiments, a topical cannabinoid composition provided herein may comprise at least 1%, at least 2%, at least 3%, at least 4%, or at least 5% (w/w) of penetration enhancer(s).

In certain exemplary, non-limiting embodiments, a topical cannabinoid composition provided herein may comprise about 1%, about 2%, about 3%, about 4%, or about 5% (w/w) of penetration enhancer(s).

In certain exemplary, non-limiting embodiments, both the humectant and the penetration enhancer of a topical cannabinoid composition provided herein have a molecular weight lower than the molecular weight of the cannabinoid. For example, the humectant and/or the penetration enhancer may have a molecular weight between 50 and 300 g/mol, between 75 and 200 g/mol, or between 75 and 150 g/mol. Without being limited by any particular theory, it is expected that the movement of compounds through skin layers, such as sub-epidermal layers, is affected by their molecular size, which is related to their molecular weight, among other factors. Therefore, it is expected to be beneficial to minimize the transport/absorption time of ingredients other than cannabinoids across epidermis into the sub-epidermal layers.

In certain exemplary, non-limiting embodiments, a topical cannabinoid composition provided herein may include a first emulsifier, such as an ethoxylated fatty alcohol, PEG-1000 monocetyl ether, alkyl trimethyl ammonium bromide, glycerol monostearate, potassium stearate, sodium lauryl sulfate, sodium cetearyl sulfate, or a saponin.

In certain exemplary, non-limiting embodiments, a topical cannabinoid composition provided herein may include about 0.5% to about 5% (w/w) of a first emulsifier.

In certain exemplary, non-limiting embodiments, a topical cannabinoid composition provided herein may comprise about 0.5%, about 1%, about 2%, about 3%, about 4%, or about 5% (w/w) of a first emulsifier.

In certain exemplary, non-limiting embodiments, a topical cannabinoid composition provided herein may include one or more further emulsifiers, such as an ethoxylated fatty alcohol, PEG-1000 monocetyl ether, alkyl trimethyl ammonium bromide, glycerol monostearate, potassium stearate, sodium lauryl sulfate, sodium cetearyl sulfate, saponin, Polysorbate 20 (Tween™ 20), Polysorbate 40 (Tween 40), Polysorbate 60 (Tween 60) and Polysorbate 80 (Tween 80), combinations, and mixtures thereof.

In certain exemplary, non-limiting embodiments, a topical cannabinoid composition provided herein may include about 1% to about 5% (w/w) of further emulsifier(s).

In certain exemplary, non-limiting embodiments, a topical cannabinoid composition provided herein may comprise about 1%, about 2%, about 3%, about 4%, or about 5% (w/w) of further emulsifier(s).

In certain exemplary, non-limiting embodiments, a topical cannabinoid composition provided herein may include one or more thickening agents, such as magnesium stearate, calcium carbonate, behenic acid, cetyl alcohol, cetearyl alcohol, cetostearyl alcohol, stearyl alcohol, combinations, and mixtures thereof.

In certain exemplary, non-limiting embodiments, a topical cannabinoid composition provided herein may include about 1% to about 10% (w/w) of thickening agent(s).

In certain exemplary, non-limiting embodiments, a topical cannabinoid composition provided herein may comprise about 1%, about 2%, about 3%, about 4%, about 5%, about 6%, about 7%, about 8%, about 9%, or about 10% (w/w) of thickening agent(s).

In certain exemplary, non-limiting embodiments, a topical cannabinoid composition provided herein may include one or more skin protecting agents, such as allantoin, aluminum hydroxide gel, calamine, cocoa butter, cod liver oil, colloidal oatmeal, hard fat, kaolin, petrolatum, topical starch, white petrolatum, zinc acetate, zinc carbonate, zinc oxide, combinations, and mixtures thereof.

In certain exemplary, non-limiting embodiments, a topical cannabinoid composition provided herein may include about 1% to about 15% (w/w) of skin protecting agent(s).

In certain exemplary, non-limiting embodiments, a topical cannabinoid composition provided herein may comprise about 1%, about 2%, about 3%, about 4%, about 5%, about 6%, about 7%, about 8%, about 9%, about 10%, about 11%, about 12%, about 13%, about 14%, or about 15% (w/w) of skin protecting agent(s).

In certain exemplary, non-limiting embodiments, a topical cannabinoid composition provided herein may include one or more emollients, such as PEG-30 glyceryl cocoate, PEG-6 caprylic/capric glyceride, sucrose cocoate, isoamyl caproate, isoamyl cocoate, polyglyceryl-3 cocoate, caprylic/capric triglyceride, decal cocoates, combinations, and mixtures thereof.

In certain exemplary, non-limiting embodiments, a topical cannabinoid composition provided herein may include about 1% to about 5% (w/w) of emollient(s).

In certain exemplary, non-limiting embodiments, a topical cannabinoid composition provided herein may comprise about 1%, about 2%, about 3%, about 4%, or about 5% (w/w) of emollient(s).

In certain exemplary, non-limiting embodiments, a topical cannabinoid composition provided herein may include one or more preservatives, such as ethylparaben, methylparaben, propylparaben, butylparaben, isobutylparaben, benzalkonium chloride, imidurea, phenoxyethanol, combinations, and mixtures thereof.

In certain exemplary, non-limiting embodiments, a topical cannabinoid composition provided herein may include about 0.1% to about 3% (w/w) of preservative(s).

In certain exemplary, non-limiting embodiments, a topical cannabinoid composition provided herein may comprise about 0.1%, about 0.5%, about 1%, about 2%, or about 3% (w/w) of preservative(s).

In certain exemplary, non-limiting embodiments, a topical cannabinoid composition provided herein may include one or more sunscreen agents, such as octyl methoxycinnamate, avobenzone, oxybenzone, titanium dioxide, zinc oxide, combinations, and mixtures thereof.

In certain exemplary, non-limiting embodiments, a topical cannabinoid composition provided herein may include about 0.1% to about 0.5% (w/w) of sunscreen agent(s).

In certain exemplary, non-limiting embodiments, a topical cannabinoid composition provided herein may comprise about 0.1%, about 0.2%, about 0.3%, about 0.4%, or about 0.5% (w/w) of sunscreen agent(s).

In certain exemplary, non-limiting embodiments, a topical cannabinoid composition provided herein may include one or more pH adjusting agents/buffering agents, such as citrate/citric acid, acetate/acetic acid, phosphate/phosphoric acid, formate/formic acid, propionate/propionic acid, lactate/lactic acid, carbonate/carbonic acid, ammonium/ammonia buffer, combinations, and mixtures thereof. In certain exemplary, non-limiting embodiments, the composition may have a pH within the range of about 5 to about 7.

In certain exemplary, non-limiting embodiments, a topical cannabinoid composition provided herein may include one or more carriers, such as water, lanolin or lanolin alcohols, mineral oil, fragrant or essential oil, combinations, and mixtures thereof.

In certain exemplary, non-limiting embodiments, a topical cannabinoid composition provided herein may retain a viscosity in a range of about 20,000 to about 100,000 cps and a pH within a range of about 5 to about 7 while being cycled weekly between freezer and ambient room temperature conditions for a minimum of 1 month.

In certain exemplary, non-limiting embodiments, a topical cannabinoid composition provided herein may, when applied to a 0.45 μm cellulose acetate membrane mounted in a Franz cell, have a cannabinoid diffusion rate of about 24 μg/cm²/h to about 33 μg/cm²/h.

In certain exemplary, non-limiting embodiments, a topical cannabinoid composition provided herein may, when applied to a membrane comprising lipid-impregnated polyethersulfone and polyolefin layers mounted in a Franz cell, has a cannabinoid diffusion rate of 0 μg/cm²/h to about 1.5 μg/cm²/h.

It is understood that the amount of cannabinoid necessary to achieve a desired therapeutic result is influenced by, and will therefore vary based on, a number of factors, including for example and without limitation, the age, sex, and weight of the subject, factors that influence the metabolic rate, and the specific conditions, diseases or related treatment symptoms of the subject. The concentration of at least one cannabinoid in compositions provided herein is between about 0.002% and about 10%.

One of skill in the art will understand that the ingredients in the final formulations must total 100% and, based on the teachings provided herein, will understand that modifications to the exemplary formulations provided herein are possible (e.g., replacement of a recited ingredient with a different ingredient, addition of a different ingredient, and/or modification of an amount of an ingredient) provided that such modifications result in a formulation as taught and described herein (i.e., capable of delivering an active agent such as a cannabinoid topically).

In another aspect, there is provided a method of applying a topical cannabinoid composition provided herein to the skin of a subject. In some embodiments, the skin is affected by a skin disease or condition. Application may be carried out by dropping, spraying, diffusing, dispersing, squirting, or spreading the composition, and may optionally be carried out using an applicator, such as a dropper, a nebulizer, an impregnated gauze sheet, a syringe, or a cotton swab.

In another aspect, there is provided a method of treating a skin disease or condition in a subject, comprising applying a topical cannabinoid composition provided herein to the skin of a subject.

In another aspect, there is provided a use of a topical cannabinoid composition provided herein for the treatment of a skin disease or condition in a subject.

In certain exemplary, non-limiting embodiments, the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoictic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacca; scabies; shingles; skin cancer; squamous cell carcinoma; Sweet's syndrome; vitiligo; or, any symptom or side effect associated with any of said skin diseases or conditions.

Still further embodiments of the present disclosure relate to providing a kit, which may include a container containing a topical cannabinoid composition provided herein, or a number of containers containing materials for preparing the topical cannabinoid composition. The kit may also include instructions for treating a skin disease or condition using the topical cannabinoid composition including dosage and how the composition may be applied to the skin. When separate containers are provided in the kit, and depending on the contents in these containers, the kit may also include instructions for preparing a topical cannabinoid composition, or compositions with different concentrations of active ingredients, from the materials included in the kit and optionally other materials such as a carrier or other additives. The kit may further include an applicator for applying the topical cannabinoid composition to the skin of a subject, and may include specific instructions on how to use the applicator.

In certain exemplary, non-limiting embodiments, a topical cannabinoid composition provided herein may be prepared or obtained from a kit comprising (a) one or more cannabinoids; (b) one or more humectants; (c) one or more penetration enhancers; (d) a liquid carrier selected for application of the topical composition to the skin of a subject; and (e) instructions, wherein at least one of (a), (b) and (c) is not mixed with (d) in the kit, and wherein the instructions comprise information allowing all of (a), (b) and (c) be mixed with (d) at selected concentrations disclosed herein. The kit may include separate containers or instructions for providing or preparing more than one composition with different concentrations for one or more of (a), (b) and (c).

In certain exemplary, non-limiting embodiments, a kit may include a container containing a topical cannabinoid composition provided herein. The composition may be in form of cream, ointment, gel, liquid or the like as described above. The container may be, for example, a liquid bottle or a paste tube depending on the physical form of the composition. In other embodiments, a kit may include a plurality of containers containing materials for forming a topical cannabinoid composition provided herein. The kit may further comprise at least one of instructions for applying the composition to skin; instructions for using the composition to treat a skin disease or condition according to the methods or uses provided herein; and instructions for using the materials in the plurality of containers to prepare the composition according to the methods of preparation provided herein. Optional components of a kit may include one or more applicators (such as droppers, sprayers, gauze sheets, and cotton-tipped applicators) for applying the composition to skin. The one or more applicators may be sterilized and contained in a sealed sterile packaging.

The discussion herein and the following Examples set forth and illustrate various exemplary embodiments of the present disclosure, which are understood to be illustrative and non-limiting.

### EMBODIMENTS

Particular embodiments of the disclosure include, without limitation, the following:
1. A topical cannabinoid composition comprising:
   a. a cannabinoid at 0.01-10% (w/w),
   b. a humectant at 0.01-10% (w/w),
   c. a penetration enhancer at 1-5% (w/w), and
   d. water to make up 100% by weight,
   wherein the topical cannabinoid composition comprises less than 40% (w/w) of ethanol.
2. The topical cannabinoid composition of embodiment 1, wherein the topical cannabinoid composition comprises less than 35%, less than 30%, less than 25%, less than 20%, less than 15%, less than 10%, less than 5%, less than 1%, less than 0.5%, or less than 0.1% (w/w) of ethanol.
3. The topical cannabinoid composition of embodiment 1 or 2, wherein the topical cannabinoid composition comprises less than 10% (w/w) of ethanol.
4. The topical cannabinoid composition of embodiment 3, wherein the topical cannabinoid composition comprises less than 1% (w/w) of the ethanol.
5. A topical cannabinoid composition comprising:
   a. a cannabinoid at 0.01-10% (w/w),
   b. a humectant at 0.01-10% (w/w),
   c. a penetration enhancer at 1-5% (w/w), and
   d. water at no less than 31% (w/w).
6. The topical cannabinoid composition of any one of embodiments 1 to 5, which comprises about 0.01% (w/w) of the cannabinoid.
7. The topical cannabinoid composition of any one of embodiments 1 to 5, which comprises about 0.1% (w/w) of the cannabinoid.
8. The topical cannabinoid composition of any one of embodiments 1 to 5, which comprises about 0.5% (w/w) of the cannabinoid.
9. The topical cannabinoid composition of any one of embodiments 1 to 5, which comprises about 1% (w/w) of the cannabinoid.
10. The topical cannabinoid composition of any one of embodiments 1 to 5, which comprises about 2% (w/w) of the cannabinoid.
11. The topical cannabinoid composition of any one of embodiments 1 to 5, which comprises about 3% (w/w) of the cannabinoid.
12. The topical cannabinoid composition of any one of embodiments 1 to 5, which comprises about 4% (w/w) of the cannabinoid.
13. The topical cannabinoid composition of any one of embodiments 1 to 5, which comprises about 5% (w/w) of the cannabinoid.
14. The topical cannabinoid composition of any one of embodiments 1 to 5, which comprises about 6% (w/w) of the cannabinoid.
15. The topical cannabinoid composition of any one of embodiments 1 to 5, which comprises about 7% (w/w) of the cannabinoid.
16. The topical cannabinoid composition of any one of embodiments 1 to 5, which comprises about 8% (w/w) of the cannabinoid.
17. The topical cannabinoid composition of any one of embodiments 1 to 5, which comprises about 9% (w/w) of the cannabinoid.
18. The topical cannabinoid composition of any one of embodiments 1 to 5, which comprises about 10% (w/w) of the cannabinoid.
19. The topical cannabinoid composition of any one of embodiments 1 to 18, wherein the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), or any combination thereof.
20. The topical cannabinoid composition of embodiment 19, wherein the cannabinoid is cannabidiol (CBD).
21. The topical cannabinoid composition of any one of the embodiments 1 to 20, which comprises about 0.01% (w/w) of the humectant.
22. The topical cannabinoid composition of any one of the embodiments 1 to 20, which comprises about 0.1% (w/w) of the humectant.
23. The topical cannabinoid composition of any one of the embodiments 1 to 20, which comprises about 0.5% (w/w) of the humectant.
24. The topical cannabinoid composition of any one of the embodiments 1 to 20, which comprises about 1% (w/w) of the humectant.
25. The topical cannabinoid composition of any one of the embodiments 1 to 20, which comprises about 2% (w/w) of the humectant.
26. The topical cannabinoid composition of any one of the embodiments 1 to 20, which comprises about 3% (w/w) of the humectant.
27. The topical cannabinoid composition of any one of the embodiments 1 to 20, which comprises about 4% (w/w) of the humectant.
28. The topical cannabinoid composition of any one of the embodiments 1 to 20, which comprises about 5% (w/w) of the humectant.
29. The topical cannabinoid composition of any one of the embodiments 1 to 20, which comprises about 6% (w/w) of the humectant.
30. The topical cannabinoid composition of any one of the embodiments 1 to 20, which comprises about 7% (w/w) of the humectant.
31. The topical cannabinoid composition of any one of the embodiments 1 to 20, which comprises about 8% (w/w) of the humectant.
32. The topical cannabinoid composition of any one of the embodiments 1 to 20, which comprises about 9% (w/w) of the humectant.
33. The topical cannabinoid composition of any one of the embodiments 1 to 20, which comprises about 10% (w/w) of the humectant.
34. The topical cannabinoid composition of any one of embodiments 1 to 33, wherein the humectant has a molecular weight lower than the molecular weight of the cannabinoid.
35. The topical cannabinoid composition of any one of embodiments 1 to 34, wherein the humectant is a polyol.
36. The topical cannabinoid composition of embodiment 35, wherein the polyol is glycerin, propylene glycol, butylene glycol, dipropylene glycol, pentylene glycol, hexylene glycol, polyethylene glycol, or any combination thereof.
37. The topical cannabinoid composition of embodiment 36, wherein the humectant is propylene glycol.
38. The topical cannabinoid composition of any one of the embodiments 1 to 37, which comprises about 1% (w/w) of the penetration enhancer.
39. The topical cannabinoid composition of any one of the embodiments 1 to 37, which comprises about 2% (w/w) of the penetration enhancer.
40. The topical cannabinoid composition of any one of the embodiments 1 to 37, which comprises about 3% (w/w) of the penetration enhancer.
41. The topical cannabinoid composition of any one of the embodiments 1 to 37, which comprises about 4% (w/w) of the penetration enhancer.
42. The topical cannabinoid composition of any one of the embodiments 1 to 37, which comprises about 5% (w/w) of the penetration enhancer.
43. The topical cannabinoid composition of any one of embodiments 1 to 42, wherein the penetration enhancer has a molecular weight lower than the molecular weight of the cannabinoid.
44. The topical cannabinoid composition of any one of embodiments 1 to 43, wherein the penetration enhancer is a glycol ether, octyldecanol, isopropyl myristate, or any combination thereof.
45. The topical cannabinoid composition of embodiment 44, wherein the penetration enhancer is a glycol ether selected from diethylene glycol monoethyl ether, steareth-20, and steareth-2, or is any combination thereof.
46. The topical cannabinoid composition of embodiment 45, wherein the penetration enhancer is diethylene glycol monoethyl ether.
47. The topical cannabinoid composition of any one of embodiments 1 to 46, wherein both the humectant and the penetration enhancer have a molecular weight lower than the molecular weight of the cannabinoid.
48. The topical cannabinoid composition of any one of embodiments 1 to 47, wherein the humectant is propylene glycol, and the penetration enhancer is diethylene glycol monoethyl ether.
49. The topical cannabinoid composition of any one of embodiments 1 to 48, wherein the composition further comprises a thickening agent at 1-10% (w/w).
50. The topical cannabinoid composition of embodiment 49, wherein the thickening agent is magnesium stearate, calcium carbonate, behenic acid, cetyl alcohol, cetearyl alcohol, cetostearyl alcohol, stearyl alcohol, or any combination thereof.
51. The topical cannabinoid composition of embodiment 50, wherein the thickening agent is cetostearyl alcohol.
52. The topical cannabinoid composition of any one of embodiments 1 to 51, wherein the composition further comprises a first emulsifier at 0.5-5% (w/w).
53. The topical cannabinoid composition of embodiment 52, wherein the first emulsifier is an ethoxylated fatty alcohol, PEG-1000 monocetyl ether, alkyl trimethyl ammonium bromide, glycerol monostearate, potassium stearate, sodium lauryl sulfate, sodium cetearyl sulfate, saponin, or any combination thereof.
54. The topical cannabinoid composition of embodiment 53, wherein the first emulsifier is glycerol monostearate.
55. The topical cannabinoid composition of any one of embodiments 1 to 54, wherein the composition further comprises a second emulsifier at 1-5% (w/w).

56. The topical cannabinoid composition of embodiment 55, wherein the second emulsifier is Polysorbate 20 (Tween 20), Polysorbate 40 (Tween 40), Polysorbate 60 (Tween 60) and Polysorbate 80 (Tween 80), or any combination thereof.
57. The topical cannabinoid composition of embodiment 56, wherein the second emulsifier is Polysorbate 60 (Tween 60).
58. The topical cannabinoid composition of any one of embodiments 1 to 57, wherein the composition further comprises a skin protectant at 1-15% (w/w).
59. The topical cannabinoid composition of embodiment 58, wherein the skin protectant is allantoin, aluminum hydroxide gel, calamine, cocoa butter, cod liver oil, colloidal oatmeal, hard fat, kaolin, petrolatum, topical starch, white petrolatum, zinc acetate, zinc carbonate, zinc oxide, or any combination thereof.
60. The topical cannabinoid composition of embodiment 59, wherein the skin protectant is white petrolatum.
61. The topical cannabinoid composition of any of embodiments 1 to 60, wherein the composition further comprises an emollient at 1-5% (w/w).
62. The topical cannabinoid composition of embodiment 61, wherein the emollient is PEG-30 glyceryl cocoate, PEG-6 caprylic/capric glyceride, sucrose cocoate, isoamyl caproate, isoamyl cocoate, polyglyceryl-3 cocoate, caprylic/capric triglyceride, a decal cocoate, or any combination thereof.
63. The topical cannabinoid composition of embodiment 62, wherein the emollient is caprylic/capric triglyceride.
64. The topical cannabinoid composition of any one of embodiments 1 to 63, wherein the composition further comprises a preservative at 0.1-3% (w/w).
65. The topical cannabinoid composition of embodiment 64, wherein the preservative is ethylparaben, methylparaben, propylparaben, butylparaben, isobutylparaben, benzalkonium chloride, imidurea, phenoxyethanol, or any combination thereof.
66. The topical cannabinoid composition of embodiment 65, wherein the preservative is phenoxyethanol.
67. The topical cannabinoid composition of any one of embodiments 1 to 66, wherein the composition further comprises a sunscreen agent at 0.1-0.5% (w/w).
68. The topical cannabinoid composition of embodiment 67, wherein the sunscreen agent is octyl methoxycinnamate, avobenzone, oxybenzone, titanium dioxide, zinc oxide, or any combination thereof.
69. The topical cannabinoid composition of embodiment 68, wherein the sunscreen agent is titanium dioxide.
70. The topical cannabinoid composition of any one of embodiments 1 to 69, wherein the composition further comprises a pH adjusting agent.
71. The topical cannabinoid composition of embodiment 70, wherein the pH adjusting agent is a buffer.
72. The topical cannabinoid composition of embodiment 71, wherein the buffer is citrate/citric acid, acetate/acetic acid, phosphate/phosphoric acid, formate/formic acid, propionate/propionic acid, lactate/lactic acid, carbonate/carbonic acid, or ammonium/ammonia buffer.
73. The topical cannabinoid composition of embodiment 72, wherein the buffer is citrate/citric acid.
74. The topical cannabinoid composition of any one of embodiments 1 to 73, wherein the composition has a pH within the range of about 5 to about 7.
75. The topical cannabinoid composition of any one of embodiments 1 to 74, wherein the composition has a viscosity in a range of about 10,000 to about 250,000 cps, about 20,000 to about 100,000 cps, about 30,000 to about 100,000 cps, about 40,000 to about 100,000 cps, about 50,000 to about 100,000 cps, about 60,000 to about 100,000 cps, or about 70,000 to about 100,000 cps.
76. The topical cannabinoid composition of any one of embodiments 1 to 75, wherein the composition has a viscosity in a range of about 20,000 to about 100,000 cps and a pH within a range of about 5 to about 7, and retains its viscosity and pH within the said ranges while being cycled weekly between freezer and ambient room temperature conditions for a minimum of 1 month.
77. The topical cannabinoid composition of any one of embodiments 1 to 76, wherein the composition, when applied to a 0.45 μm cellulose acetate membrane mounted in a Franz cell, has a cannabinoid diffusion rate of about 24 μg/cm²/h to about 33 μg/cm²/h.
78. The topical cannabinoid composition of any one of embodiments 1 to 77, wherein the composition, when applied to a membrane comprising lipid-impregnated polyethersulfone and polyolefin layers mounted in a Franz cell, has a cannabinoid diffusion rate of 0 μg/cm²/h to about 1.5 μg/cm²/h.
79. The topical cannabinoid composition of any one of embodiments 1 to 78, wherein the composition is a cream, ointment, gel, lotion, liquid, solution, spray, aerosol, any other dosage form suitable for topical application, or any combination thereof.
80. The topical cannabinoid composition of embodiment 79, wherein the composition is a cream.
81. A method comprising applying the topical cannabinoid composition of any one of embodiments 1 to 80 to the skin of a subject.
82. The method of embodiment 81, wherein the skin of the subject is affected by a skin disease or condition.
83. The method of embodiment 82, wherein the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; skin cancer; squamous cell carcinoma; Sweet's syndrome; vitiligo; or, any symptom or side effect associated with any of said skin diseases or conditions.
84. The method of embodiment 83, wherein the skin disease or condition is eczema, psoriasis, epidermolysis bullosa, or skin cancer.
85. The method of embodiment 84, wherein the skin disease or condition is epidermolysis bullosa.
86. The method of embodiment 82, wherein the skin condition is a blister, rash, sore, ulcer, or wound.
87. The method of embodiment 82, wherein the skin is broken, chapped, cracked, dry, itchy, painful, sensitive, swollen, or tender.
88. A method of treating a skin disease or condition in a subject, comprising applying the topical cannabinoid compositions of any one of embodiments 1 to 80 to the skin of a subject.
89. The method of embodiment 88, wherein the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; skin cancer; squamous cell carcinoma; Sweet's syndrome; vitiligo; or, any symptom or side effect associated with any of said skin diseases or conditions.
90. The method of embodiment 89, wherein the skin disease or condition is eczema, psoriasis, epidermolysis bullosa, or skin cancer.
91. The method of embodiment 90, wherein the skin disease or condition is epidermolysis bullosa.
92. The method of embodiment 88, wherein the skin condition is a blister, rash, sore, ulcer, or wound.
93. The method of embodiment 88, wherein the skin is broken, chapped, cracked, dry, itchy, painful, sensitive, swollen, or tender.
94. The method of any one of embodiments 81 to 93, wherein the applying comprises dropping, spraying, diffusing, dispersing, squirting, or spreading the composition.
95. The method of any one of embodiments 81 to 94, wherein the subject is a mammal.
96. The method of any one of embodiments 81 to 95, wherein the subject is a companion animal.
97. The method of embodiment 95, wherein the subject is a human.
98. The method of any one of embodiments 81 to 97, wherein the method further comprises administering an additional therapy for a skin disease or condition.
99. Use of the topical cannabinoid formulation of any one of embodiments 1 to 80 for the treatment of a skin disease or condition in a subject.
100. The use of embodiment 99, wherein the skin disease or condition is: microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacca; scabies; shingles; skin cancer; squamous cell carcinoma; Sweet's syndrome; vitiligo; or, any symptom or side effect associated with any of said skin diseases or conditions.
101. The use of embodiment 100, wherein the skin disease or condition is eczema, psoriasis, epidermolysis bullosa, or skin cancer.
102. The use of embodiment 101, wherein the skin disease or condition is epidermolysis bullosa.
103. The use of embodiment 99, wherein the skin condition is a blister, rash, sore, ulcer, or wound.
104. The use of embodiment 99, wherein the skin is broken, chapped, cracked, dry, itchy, sensitive, swollen, or tender.
105. The use of any of embodiments 99 to 104, wherein the subject is a mammal.
106. The use of any of embodiments 99 to 105, wherein the subject is a companion animal.
107. The use of embodiment 105, wherein the subject is a human.
108. The use of any one of embodiments 99 to 107, wherein the treatment further comprises an additional therapy for a skin disease or condition.
109. A kit comprising a container containing the topical cannabinoid composition of any one of embodiments 1 to 80.
110. A kit comprising a plurality of containers containing materials for forming the topical cannabinoid composition of any one of embodiments 1 to 80.
111. The kit of embodiment 109 or 110, further comprising instructions for preparing the topical cannabinoid composition from the materials in the containers.
112. The kit of any one of embodiments 109 to 111, further comprising instructions for applying the topical cannabinoid composition to the skin of a subject.
113. The kit of any one of embodiments 109 to 112, further comprising instructions for using the topical cannabinoid composition to treat a skin disease or condition according to the method of any one of embodiments 81 to 98.
114. The kit of any one of embodiments 109 to 113, further comprising one or more applicators for applying the topical cannabinoid composition to the skin of a subject.
115. A topical cannabinoid composition comprising:
   a) a cannabinoid at 0.01-10% (w/w),
   b) a humectant at 0.01-10% (w/w),
   c) a penetration enhancer at 1-5% (w/w),
   d) a first emulsifier at 0.5-5% (w/w),
   e) a thickening agent at 1-10% (w/w),
   f) a skin protectant at 1-15% (w/w),
   g) an emollient at 1-5% (w/w),
   h) a preservative at 0.1-3% (w/w),
   i) a sunscreen agent at 0.1-0.5% (w/w),
   j) a second emulsifier at 1-5% (w/w),
   k) a pH adjusting agent in a quantity sufficient for the composition to maintain pH 5-7, and
   l) water to make up 100% by weight.
116. The topical cannabinoid composition of embodiment 115, wherein said cannabinoid cannabidiol (CBD), cannabidiolic acid (CBDA), cannabidivarin (CBDV), cannabigerol (CBG), cannabigervarin (CBGV), tetrahydrocannabivarin (THCV) or tetrahydrocannabinol (THC).
117. The topical cannabinoid composition of embodiment 116, wherein said cannabinoid is CBD.
118. The topical cannabinoid composition of embodiment 115, wherein said humectant is glycerin, propylene glycol, butylene glycol, dipropylene glycol, pentylene glycol, hexylene glycol or polyethylene glycol.
119. The topical cannabinoid composition of embodiment 118, wherein said humectant is propylene glycol.
120. The topical cannabinoid composition of embodiment 115, wherein said penetration enhancer is diethylene glycol monoethyl ether, steareth-20, steareth-2, octyldecanol or isopropyl myristate.
121. The topical cannabinoid composition of embodiment 120, wherein said penetration enhancer is diethylene glycol monoethyl ether.
122. The topical cannabinoid composition of embodiment 115, wherein said first emulsifier is ethoxylated fatty alcohols, PEG-1000 monocetyl ether, alkyl trimethyl ammonium bromide, glycerol monostearate, potassium stearate, sodium lauryl sulfate, sodium cetearyl sulfate or saponins.
123. The topical cannabinoid composition of embodiment 122, wherein said first emulsifier is glycerol monostearate.
124. The topical cannabinoid composition of embodiment 115, wherein said thickening agent is magnesium stearate, calcium carbonate, behenic acid, cetyl alcohol, cetearyl alcohol, cetostearyl alcohol or stearyl alcohol.
125. The topical cannabinoid composition of embodiment 124, wherein said thickening agent is cetostearyl alcohol.
126. The topical cannabinoid composition of embodiment 115, wherein said skin protectant is allantoin, aluminum hydroxide gel, calamine, cocoa butter, cod liver oil, colloidal oatmeal, hard fat, kaolin, petrolatum, topical starch, white petrolatum, zinc acetate, zinc carbonate or zinc oxide.
127. The topical cannabinoid composition of embodiment 126, wherein said skin protectant is white petrolatum.
128. The topical cannabinoid composition of embodiment 115, wherein said emollient is PEG-glyceryl cocoate, PEG-6 caprylic/capric glyceride, sucrose cocoate, isoamyl caproate, isoamyl cocoate, polyglyceryl-3 cocoate, caprylic/capric triglyceride or decal cocoates.
129. The topical cannabinoid composition of embodiment 128, wherein said emollient is caprylic/capric triglyceride.
130. The topical cannabinoid composition of embodiment 115, wherein said preservative is ethylparaben, methylparaben, propylparaben, butylparaben, isobutylparaben, benzalkonium chloride, imidurea or phenoxyethanol.
131. The topical cannabinoid composition of embodiment 130, wherein said preservative is phenoxyethanol.
132. The topical cannabinoid composition of embodiment 115, wherein said sunscreen agent is octyl methoxycinnamate, avobenzone, oxybenzone, titanium dioxide or zinc oxide.
133. The topical cannabinoid composition of c embodiment 132, wherein said sunscreen agent is titanium dioxide.
134. The topical cannabinoid composition of embodiment 115, wherein said second emulsifier is Polysorbate 20 (Tween 20), Polysorbate 40 (Tween 40), Polysorbate 60 (Tween 60) or Polysorbate 80 (Tween 80).
135. The topical cannabinoid composition of embodiment 134, wherein said second emulsifier is Polysorbate 60 (Tween 60).
136. The topical cannabinoid composition of embodiment 115, wherein said pH adjusting agent is a buffer.
137. The topical cannabinoid composition of embodiment 136, wherein said buffer is citrate/citric acid, acetate/acetic acid, phosphate/phosphoric acid, formate/formic acid, propionate/propionic acid, lactate/lactic acid, carbonate/carbonic acid and ammonium/ammonia buffer.
138. The topical cannabinoid composition of embodiment 137, wherein said buffer is citrate/citric acid.
139. The topical cannabinoid composition of embodiment 115, wherein the said composition has a pH within the range of about 5 to 7.
140. The topical cannabinoid composition of embodiment 115, wherein the said composition has a viscosity in a range of about 20,000 to 100,000 cps.
141. The topical cannabinoid composition of embodiment 115, wherein the said composition has a very low rate of transdermal diffusion as measured using in-vitro Franz diffusion cell assays.
142. The topical cannabinoid composition of embodiment 115, wherein the said composition has very strong localized retention and very low systemic absorption.
143. The topical cannabinoid composition of embodiment 115, wherein the said composition is a topical cream composition.
144. The topical cannabinoid composition of any one of embodiments 115 to 143, wherein the compositions are useful for the treatment of one or more skin diseases or conditions such as microbial infection-induced dermatitis; solar dermatitis; atopic dermatitis; contact dermatitis; acne; alopecia areata; basal cell carcinoma; Bowen's disease; congenital erythropoietic porphyria; Darier's disease; epidermolysis bullosa; eczema; erythropoietic protoporphyria; fungal infections of nails; Hailey-Hailey disease; herpes simplex; hidradenitis suppurativa; hirsutism; hyperhidrosis; ichthyosis; impetigo; keloids; keratosis pilaris; lichen planus; lichen sclerosus; melisma; pemphigus vulgaris; plantar warts (verrucas); pityriasis lichenoides; polymorphic light eruption; psoriasis; pyoderma gangrenosum; rosacea; scabies; shingles; skin cancer; squamous cell carcinoma; Sweet's syndrome; vitiligo; or, any symptom or side effect associated with any of said skin diseases or conditions.
145. A cream comprising:
   a. a cannabinoid at 0.01-10% (w/w),
   b. a cannabinoid localizing agent at 1-15% (w/w), and
   c. water to make up 100% by weight,
146. The cream of embodiment 145, wherein the composition has a viscosity in a range of about 20,000 to about 100,000 cps.

### EXAMPLES

### Example 1: 3% CBD Composition

TABLE 1
Ingredients of Composition 1.

| Component | % w/w |
|---|---|
| CBD | 3.0 |
| Propylene Glycol | 1.0 |
| Diethylene glycol monoethyl | 3.0 |
| ether | |
| Glycerol monostearate | 1.0 |
| Cetostearyl Alcohol | 5.0 |
| White Petrolatum | 8.0 |
| Triglyceride | 3.0 |
| Phenoxyethanol | 1.0 |
| Citrate Na⁺ | 0.7 |
| Titanium Dioxide | 0.2 |
| Tween 60 | 3.0 |
| 5% Citric Acid Stock Aq Soln | qs pH 5-7 |
| Purified Water | QSAD 100 |

The following procedure was used to produce a laboratory batch according to the formula in Table 1:
a) Combine CBD (molecular weight: 314.46 g/mol) and propylene glycol (molecular weight: 76.09 g/mol) in beaker. Heat to 60-70° C.
b) Combine glycerol monostearate, cetostearyl alcohol and white petrolatum in beaker. Heat to 60-70° C. Add diethylene glycol monoethyl ether (molecular weight: 134.175 g/mol), triglyceride, phenoxyethanol and triglyceride under continual mixing/heating. Mix to disperse.
c) Add the materials from step a) to the materials from step b) at 60-70° C. with rapid propeller mixing.
d) Combine water, citrate sodium salt, titanium dioxide and Tween 60. Adjust the pH with 5% citric acid. Heat to 60-70° C.
e) Add the materials from step c) to the materials from step d) at 60-70° C. with rapid propeller mixing. Mix until smooth, homogeneous cream forms.
f) Continue mixing while cooling to room temperature.

### Example 2: Assessment of Stability

The composition described in Example 1 was tested for long-term stability for up to 24 months as per ICH guidelines. The physical appearance was monitored for compliance with the specification of “smooth off-white to light yellow/light green cream”. Both pH and viscosity were monitored during the stability study. pH was measured using a standardized pH meter and 1:10 dilution of the cream in distilled water. The viscosity was determined with Brookfield Viscometer using a flat spindle CPA52Z at a speed of 0.1-4 RPM at room temperature. After one month of weekly cycling between freezer and ambient room temperature conditions, the composition remained stable. When stored at room temperature for 6 months and 24 months, the composition also remained stable. The test results are summarized in Table 2:

TABLE 2
Summary of stability test results.

| | | | 1-month | | |
|---|---|---|---|---|---|
| | | | freeze/ | | |
| | Specification | | thaw | | 24 |
| | smooth off- | Initial | cycling | 6 months | months |
| Description | white cream | complies | complies | complies | complies |
| pH | 5 to 7 | 7 | 6.3 | 6.0 | 5.0 |
| Viscosity | 20,000 to | 70,000 to | 60,000 to | 20,000 to | 20,000 to |
| (centipoise) | 100,000 | 100,000 | 100,000 | 100,000 | 100,000 |

The compositions described in this disclosure tend to remain stable against phase separation and product degradation over a wide range of storage conditions. For example, the cream composition as described above may remain stable over a temperature range of at least from about-20 to 50° C., over periods of several months, depending on the storage temperature and at accelerated conditions, i.e., after subsequent centrifugation at 2000-4000 RPM. It will be understood by one of ordinary skill in the art that stability of a composition to phase separation will be influenced by the conditions under which the composition was formed and stored.

### Example 3: Skin Permeation Testing

Skin diffusion/permeation for the composition described in Example 1 was investigated using the validated Franz cells and equipment having receptor and donor compartments using saturated solution across synthetic membranes. Synthetic membrane used was 0.45 μm Whatman™ cellulose acetate (CA) for quality control testing of batch to batch reproducibility. CA is a simple, porous synthetic membrane for assessing topical formulation that acts as a barrier with no rate-limiting property.

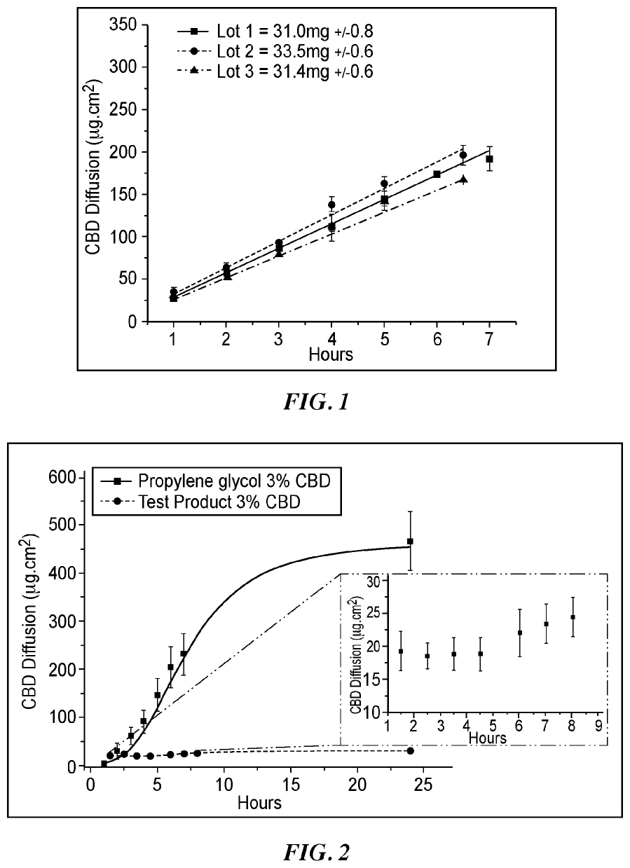

The receptor media was stirred by a magnetic stir-bar at 600 rpm. About 30-32 mg of three samples of the composition of Example 1 were weighed and added to the donor compartment. The two compartments were separated by a cellulose acetate membrane filter (0.45 μm from Whatman). The assay was carried out at 32° C. and lasted six hours. Approximately 0.2 mL of receptor media was collected every hour, and the receptor compartment was immediately refilled with fresh media. The CBD concentration in the receptor was analyzed by a developed and validated HPLC method. CBD diffusion remained similar for different samples of the composition of Example 1 showing good reproducibility as depicted in FIG. 1.

The test for evaluating transdermal diffusion was conducted using the Franz cell model according to the method described in USP 1724 (2015) SemiSolid Drug Products and using Strat-M™ membranes (EMD Millipore) and quantified by HPLC. The Strat-M™ membranes are synthetic polymeric membranes composed of two layers of polyethersulfone (PES, more resistant to diffusion) on top of one layer of polyolefin (more open and diffusive). These polymeric layers create a porous structure with a gradient across the membrane in terms of pore size and diffusivity. The porous structure is impregnated with a proprietary blend of synthetic lipids, imparting additional skin-like properties to the synthetic membrane. Thus, the Strat-M™ membranes create “morphology” resembling that observed in human skin and are used for transmembrane diffusion tests, which simulate the diffusion of various types of compounds and formulations across the human epidermis.

Very low transdermal diffusion rates of CBD comprised in the composition of Example 1 were observed as compared to the positive control formulation consisting of 3% (w/w) of CBD in liquid propylene glycol (FIG. 2), supportive of the claim that the cream was not absorbed systemically in a therapeutically effective concentration, thereby providing strong localized effects for longer duration. It remained in the epidermal layer as required for the treatment of skin diseases or conditions.

### Example 4: Quantification Analysis

The composition described in Example 1 was tested for quantification of CBD in the final formulation. Working calibration standards of CBD were prepared at concentrations of 100, 50, 10, 5, 1, and 0.5 μg/mL in acetonitrile. Standards were stored as recommended by the supplier.

Analysis was performed using Agilent™ 1260 Infinity II (Agilent Technologies, Santa Clara, CA) LC system equipped with a quaternary pump, autosampler, thermo column compartment and UV DAD detector. Chemstation™ software was used to control the instrument components and to acquire, store and analyse UV data. Injection of standards and samples was set up at 20 μL.

The column chosen for CBD analysis was from Waters-Xterra™ C₁₈ 4.6×250 mm×5.0 μm (Part No 186000494) and a guard column Xterra™ (Part No186000662). The mobile phases consisted 20% of distilled water with 0.1% TFA (mobile phase A), and 80% acetonitrile with 0.1% TFA (v/v) (mobile phase B). Flow rate used was 1 mL·min⁻¹ and runtime per analysis was setup at 8 min. CBD was detected at 6.4 min. The Amax of the cannabidiol is ranging from 218 nm to 230 nm. UV-DAD data was collected at 220 nm.

The composition was transferred in a vial (20 mL) and a known volume of acetonitrile was added to the cream. The vial was vortexed (20 sec), sonicated (20 sec) and the procedure was repeated 3 times. The solution was transferred in centrifugation tube and centrifuged for 10 minutes at 4000 rpm. The supernatant was diluted, and the solution was transferred in a vial for HPLC analysis. In addition, placebo creams (no CBD) were used to confirm the efficiency/recovery of CBD extraction. A known amount of CBD (isolate/crystals) was added to a known amount of placebo cream, vortexed and a volume of acetonitrile was added; same procedure of extraction was performed.

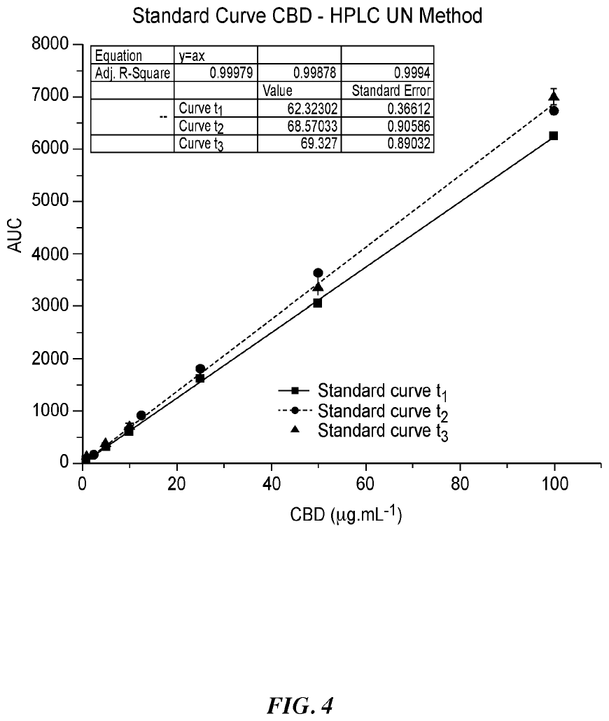

Quantification of CBD was validated through calibration curves (FIG. 4) repeated in duplicate at 3 interval times (n=3). Average slope value was determined at 66.99 with a standard deviation of 4.4 (S.D=6%). Linearity of the CBD standard curve remained at R²=0.9989.

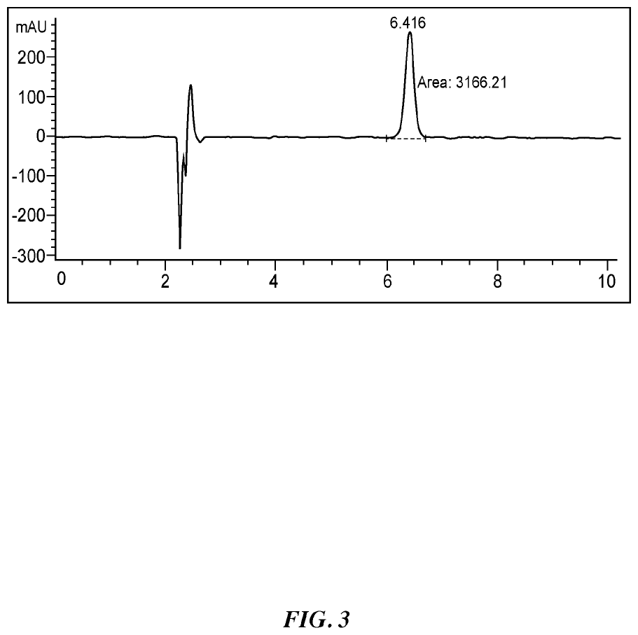

HPLC analytical method for the quantification recovered higher than 98% and lower than 102% of CBD. After 2 months storage at room temperature, extraction efficiency remained the same and no apparent drug degradation was observed by HPLC. Chromatogram of the analysis of CBD and its validation through standard curves are depicted in FIGS. 3 and 4, respectively.

### Example 5: In Vivo Pharmacology Studies

TABLE 3
Summary of in vivo pharmacology studies performed

| | Number/ | | | | | Duration of |
|---|---|---|---|---|---|---|
| | sex of | | | | | post- |
| Species | animals | Unit | Dose | Route of | Duration | exposure |
| tested | per group | dose | interval | administration | of dosing | follow-up |
| Sprague | 12/ | 15 mg/kg | Twice | Topical | 7 days | 168 hours |
| Dawley | Female | | daily | (Cream) | | |
| Rats | | | | | | |

### Methodology

### a) Animals

12 Female Sprague Dawley Rats (150-200 g) were purchased from Charles River Laboratories (Wilmington, MA). Rats were allowed to acclimatise for one week prior to commencement of the study.

### b) Treatment

At day 0, rats were anaesthetised using 3% isoflurane via inhalation. The skin on the dorsal side of the rats was shaved and a circular area of approximately 18.09 cm² was marked onto the surface of the skin. Twice daily, cream was applied to the dorsal surface of the skin using a 1 ml syringe that was prefilled with the composition described in Example 1 at a dose of 15 mg/kg of cannabidiol (CBD). Following the application of the cream and in order to prevent the cream from being removed, dermal application jackets were applied to each of the rats. These were maintained on the animals for the duration of the study (i.e. 7 days). Every 24 hours, blood was collected by puncturing the saphenous vein with a 20 G needle. At each time point, approximately 200 μL of blood was collected into Microvette™ 200 ZGel collection tubes (Sarstedt). Blood samples were centrifuged at 1.5×104 RPM for 5 mins. Following this, plasma was isolated and stored at −80° C. until the end of the study when all samples were analysed simultaneously. At the end of the study (T=168 h), rats were euthanized under anaesthetic (with 3% isoflurane via inhalation) by cardiac puncture followed by cervical dislocation. The following organs were collected; skin application site, kidneys, liver and spleen. Samples were fixed in formalin and stored in 70% ethanol prior to processing.

### c) Plasma Extraction

Plasma (20 μL) was added to a 1:1 (v/v) solution of acetonitrile:ethyl acetate at a ratio of 1:10 (plasma:organic solvent). Samples were vortexed for 30 seconds. Following this, samples were centrifuged at 1×104 RCF at 10° C. for 20 minutes. Following this the supernatant was collected and transferred to a fresh 2 mL tube. The supernatant was evaporated under nitrogen at 37° C. The samples were reconstituted with LC-MS grade methanol prior to analysed by mass spectrometry.

### d) CBD Analysis by LC-MS

LC analyses were performed on an Agilent 1260 LC infinity system consisting of a binary pump, a thermostated autosampler and a thermostated column compartment (Agilent, Waldbronn, Germany). Samples were analyzed on an Agilent Poroshell 120 C18 column (150×2.1 mm; 1.9 μm particle size) (Agilent, Waldbronn, Germany). The mobile phase was composed of (A) water w/0.1% (v/v) formic acid (FA) and (B) Acetonitrile w/0.1% (v/v) FA at a ratio of 20:80 respectively. The flow rate was 0.2 mL/min and the column temperature was maintained at 40° C. The injection volume was 5 μL and the injector needle was washed with methanol and the autosampler was maintained at room temperature.

Tandem mass spectrometry was performed using a TSQ Endura™ Triple Quadrupole Mass Spectrometer (Thermo Fisher Scientific, Waltham, MA) operating in electrospray ionization (ESI). Xcalibur software (Thermo Fisher Scientific) was used for instrument control, data acquisition, qualitative and quantitative data analyses. Cannabinoid standards (using a previously extracted CBD isolate) were prepared in methanol from a concentration range between 5-250 ng/mL. The MS conditions were set as follows: the flow rate of sheath gas, aux gas, and sweep gas were kept at 2.42 arb, 7.31 arb, and 5.69 arb, respectively. Ion transfer tube and vaporizer temperatures were set to 275° C. and 400° C. respectively. LC-MS/MS optimized parameters for CBD are reported in Table 4.

TABLE 4
LC-MS/MS parameters optimized for CBD analysis

| | | Retention | | | |
|---|---|---|---|---|---|
| | | Time | Precursor | Product | Col. |
| Analyte | Polarity | (min) | (m/z) | (m/z) | Energy |
| Cannabidiol | Positive | 6.1-6.2 | 315 | 193.315 | 24.250 |

### Results

### a) Pharmacokinetic Profile of CBD Following Application of Topical Formulation

The plasma concentration profile of CBD is presented in FIG. 5. Peak plasma concentration was achieved at 96 hours (21.08+/−3.54 ng/ml). Interestingly following this time point, plasma concentrations of CBD continued to decline for the duration of the study period, falling below the LOQ by 168 h.

### b) Monitoring of Animal Body Weight

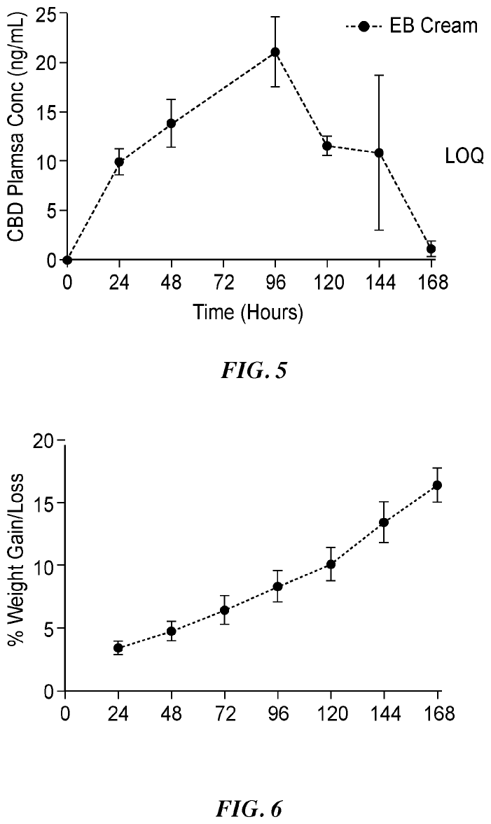

There was no appreciable reduction in the body weight of the test animals for the duration of the study period (FIG. 6). In fact, the increases in body weight that were observed during the study duration (i.e. approximately 16% in 7 days) were not dissimilar than those reported by the supplier for rats of the same age, sex and strain (InVivos; http://www.invivos.com.sg/sd/).

### c) Macroscopic Analysis of Application Site

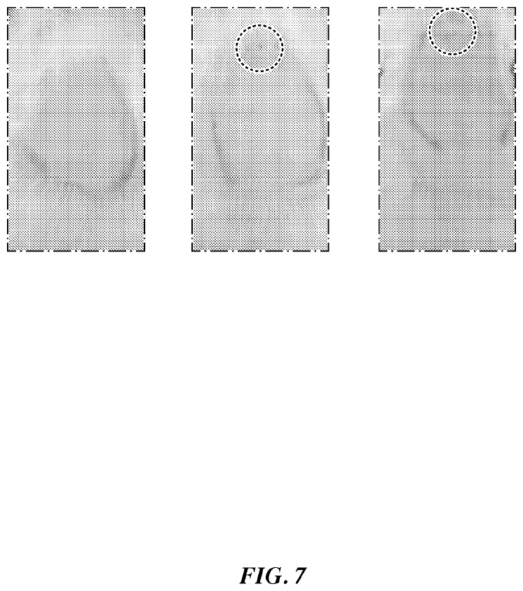

Examination of the application site on the dorsal surface of the rat at the endpoint of the study (i.e. T=168 hours) revealed no discernible or obvious signs of irritation or skin damage. There was minor irritation/pinching at the top of the application site, but this was due to the dermal application jackets that were used in this study (FIG. 7).

### d) Pathology Report on Major Organs and Skin Application Site

Histopathology was requested to assess for adverse local and systemic effects of topical treatment with product (claim 1) twice daily for 7 days in rats. Test article was applied to the dorsum over an area of approximately 18 square centimeters. Rats were sacrificed at 7 days. Microscopic findings were graded on a scale of: None=0; Minimal=1; Mild=2; Moderate=3; Marked=4.

The following criteria were used for grading:
0—Sections were histologically unremarkable.
1—Findings were barely detectable.
2—Findings were easily seen, but enveloped a small portion of the sections, for
example, up to 10%.
3—Findings were prominent and involved from 10% to 50% of the sections.
4—Findings were extensive and involved from 50% to 100% of the sections.

There was no inherent toxicity observed in any of the major organs as a consequence of the treatment under investigation. With regards to possible toxicological relevance, one animal demonstrated signs of mild focal epidermal and follicular hyperplasia and compact orthokeratotic hyperkeratosis in conjunction with mild superficial and deep neutrophilic and histiocytic perivascular inflammation in the skin. However, this was hypothesized to be due to irritation as a consequence of the dermal jackets used, not of the treatment itself.

While the foregoing has presented specific embodiments of the present disclosure, it is to be understood that these embodiments have been presented by way of example only. It is expected that others skilled in the art will perceive variations which, while varying from the foregoing, do not depart from the spirit and scope of the disclosure herein.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this disclosure belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the disclosure herein.

1. A topical cannabinoid composition comprising:
a. a cannabinoid at 0.01-10% (w/w),
b. a humectant at 0.01-10% (w/w),
c. a penetration enhancer at 1-5% (w/w), and
d. water to make up 100% by weight,
wherein the topical cannabinoid composition comprises less than 40% (w/w) of ethanol.

2. The topical cannabinoid composition of claim 1, wherein the topical cannabinoid composition comprises no more than 10% (w/w) of ethanol.

3. The topical cannabinoid composition of claim 1, wherein the topical cannabinoid composition comprises no more than 1% (w/w) of ethanol.

4. A topical cannabinoid composition comprising:
a. a cannabinoid at 0.01-10% (w/w),
b. a humectant at 0.01-10% (w/w),
c. a penetration enhancer at 1-5% (w/w), and
d. water at no less than 31% (w/w).

5. The topical cannabinoid composition of claim 1, wherein at least one of the humectant and the penetration enhancer has a molecular weight lower than the molecular weight of the cannabinoid.

6. The topical cannabinoid composition of claim 5, wherein the humectant is propylene glycol, and the penetration enhancer is diethylene glycol monoethyl ether.

7. The topical cannabinoid composition of claim 1, wherein the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), or any combination thereof.

8. The topical cannabinoid composition of claim 7, wherein the cannabinoid is cannabidiol (CBD).

9. The topical cannabinoid composition of claim 1, wherein the composition further comprises a thickening agent at 1-10% (w/w).

10. The topical cannabinoid composition of claim 9, wherein the thickening agent is cetostearyl alcohol.

11. The topical cannabinoid composition of claim 1, which has a pH of 5-7.

12. The topical cannabinoid composition of claim 1, wherein the composition further comprises one or more of:
a. a first emulsifier at 0.5-5% (w/w),
b. a skin protectant at 1-15% (w/w),
c. an emollient at 1-5% (w/w),
d. a preservative at 0.1-3% (w/w),
e. a sunscreen agent at 0.1-0.5% (w/w),
f. a second emulsifier at 1-5% (w/w),
g. a pH adjusting agent in a quantity sufficient for the composition to maintain a pH of 5-7, and
h. water to make up 100% by weight.

13. The topical cannabinoid composition of claim 12, wherein the thickening agent is cetostearyl alcohol, the first emulsifier is glycerol monostearate, the skin protectant is white petrolatum, the emollient is caprylic/capric triglyceride, the preservative is phenoxyethanol, the sunscreen agent is titanium dioxide, the second emulsifier is Polysorbate 60, and the pH adjusting agent is citrate/citric acid.

14. The topical cannabinoid composition of claim 1, wherein the composition is a cream, ointment, gel, lotion, liquid, solution, spray, aerosol, any other dosage form suitable for topical application, or any combination thereof.

15. The topical cannabinoid composition of claim 14, wherein the composition is a cream.

16. A cream composition comprising:
a. a cannabinoid at 0.01-10% (w/w),
b. a cannabinoid localizing agent at 1-15% (w/w), and
c. water to make up 100% by weight.

17. The cream of claim 16, wherein the composition has a viscosity in a range of about 20,000 to about 100,000 cps.

18-22. (canceled)
